(19) United States
(12) Patent Application Publication
(10) Pub. No.: US 2021/0283071 A1
(43) Pub. Date: Sep. 16, 2021

(54) PREPARATION OF A SINGLE-SURFACTANT MICROEMULSION

(71) Applicant: The Rosano Family Irrevocable Trust of 2015, Oradell, NJ (US)

(72) Inventor: Henri Rosano, Oradell, NJ (US)

(21) Appl. No.: 17/248,303

(22) Filed: Jan. 19, 2021

Related U.S. Application Data
(63) Continuation-in-part of application No. 16/895,897, filed on Jun. 8, 2020, now abandoned.
Application No. 16/895,897 is a continuation of application No. 16/656,716, filed on Oct. 18, 2019, now abandoned.
(60) Provisional application No. 62/747,326, filed on Oct. 18, 2018.

(51) Int. Cl. A61K 31/08; A61K 9/107; A61K 47/06; A61K 47/26

(52) U.S. Cl. CPC A61K 31/08; A61K 47/26; A61K 47/06; A61K 9/1075

(57) ABSTRACT

The present invention relates to a single surfactant microemulsion using a perfluorocarbon surfactant with low toxicity.

This application is a continuation-in-part of U.S. patent application Ser. No. 16/895,897, filed Jun. 8, 2020, which is a continuation of U.S. patent application Ser. No. 16/656,716, filed Oct. 18, 2019, which claims the benefit of U.S. Provisional Application No. 62/747,326, filed Oct. 18, 2018, which is hereby incorporated by reference.

### FIELD OF THE INVENTION

The present invention relates to new methods of preparation of microemulsions using a single surfactant. This preparation was realized using a low toxicity surfactant. It can be used as a blood substitute in humans, animals, for drug delivery, aquaculture, and other applications.

### BACKGROUND OF THE INVENTION

According to the New York Blood Center, one in three people will need a blood transfusion at some point in their lives. Real blood is not always immediately available because it has a finite shelf life (about 42 days for red blood cells) and it requires constant refrigeration. Moreover, donor blood must be compatible with the recipient's blood type.

To address this need, various blood substitutes, including oxygen therapeutics, have been proposed over the past few decades. Oxygen therapeutics mimic mammalian blood's oxygen transport ability. The transport mechanism can be based on perfluorocarbon or hemoglobin (Squires J. E., Science (2002) 295:1002-1005 and Inayat et al., Transfusion and Apheresis Science (2006) 34:25-32).

Perfluorocarbons are known to be chemically and biologically inert substances which are capable of dissolving large volumes of gases, including oxygen and carbon dioxide, at concentrations much greater than water, saline and plasma. Thus, perfluorocarbons can be a convenient means to deliver high levels of oxygen or other therapeutic gases to tissues and organ systems. As a result of their unique properties, perfluorocarbons have emerged as leading candidates for gas-transporting components in the treatment of hypoxia secondary to many acute medical situations (Spahn, 1999; U.S. Patent Application Publication No. 2009-0202617).

A promising new application of artificial oxygen carriers is maintaining tissue oxygen levels in bioreactors. Moreover, the challenge of exchanging oxygen, nutrients, and wastes typically limits the thickness of engineered tissues to a few hundred micrometers. Therefore, improved artificial oxygen carriers and methods of using such carriers continue to be sought.

Perfluorocarbons that are commonly used in medical research are biologically inert, biostatic liquids at room temperature with densities of about 1.5-2.0 g/mL and high solubilities for oxygen and carbon dioxide. However, neat perfluorocarbon liquids are unsuitable for injection into the blood stream because their hydrophobicity makes them immiscible in blood. Transportation of neat perfluorocarbon liquid into small blood vessels may cause vascular obstruction and death. Therefore, perfluorocarbons must be dispersed in physiologically acceptable aqueous emulsions for medical uses which require intravascular injection. See, e.g., L. C. Clark, Jr. et al., “Emulsions of Perfluorinated Solvents for Intravascular Gas Transport”, Fed. Proc., 34(6), pp. 1468-77 (1975); K. Yokoyama et al., “A Perfluorochemical Emulsion As An Oxygen Carrier”, Artif. Organs (Ceve), 8(1), pp. 34-40 (1984); and U.S. Pat. Nos. 4,110,474 and 4,187,252. Additionally, perfluorocarbons can result in accumulation of perfluorocarbon oil in the liver and spleen of the human recipient.

U.S. Pat. Nos. 4,722,904, 5,514,720, 5,684,050, 5,635,539, 5,171,755, 5,407,962 and 5,536,753 and U.S. Patent Publication No. 2014/0066522 disclose various emulsions of fluorinated compounds including perfluorocarbons.

U.S. Pat. No. 4,146,499 describes a method for preparing a microemulsion containing two surfactants.

The interfacial stoichioemistry of a microemulsion system was previously investigated.

There is a continuing demand for perfluorocarbon microemulsions that have low toxicity and do not result in the accumulation of perfluorocarbon in the liver or spleen.

### SUMMARY OF THE INVENTION

One embodiment of the present invention is a method of preparing a microemulsion with a single surfactant molecule (e.g., a single polyfluorocarbon surfactant), one end of which is soluble in the oil phase and the other end soluble in the aqueous phase. The single surfactant microemulsion exhibits enhanced stability compared to many microemulsions prepared with two-surfactant systems (a surfactant and a co-surfactant). The method comprises the steps of (a) mixing a surfactant and an aqueous medium, (b) adding oil, such as a perfluorocarbon (e.g. perflubron), to form the microemulsion, where the surfactant is soluble in both the oil and aqueous phases. In one embodiment, the microemulsion is used as a blood substitute for human subjects or animals, such as dogs and cats.

One embodiment is a microemulsion of perfluorocarbon (such as perflubron (also known as perfluorooctylbromide, or PFOB) or perfluorodecalin (perdecalin)) which includes a single surfactant system. The microemulsion comprises perfluorocarbon dispersed within a continuous liquid phase and a single surfactant. The microemulsions of the present invention are useful as blood substitutes and oxygen therapeutics.

One embodiment is a microemulsion comprising (a) perfluorocarbon, (b) aqueous medium; and (c) a surfactant (e.g., a non-ionic surfactant) (preferably with known low toxicity), such as a fatty alcohol surfactant or a polyfluorocarbon surfactant (e.g., a nonionic fatty alcohol surfactant or a nonionic polyfluorocarbon surfactant). In one preferred embodiment, the microemulsion includes no surfactant other than a polyfluorocarbon surfactant. In another preferred embodiment, the microemulsion includes only one polyfluorocarbon surfactant. In yet another embodiment, the microemulsion includes no surfactant other than a nonionic fatty alcohol surfactant. In yet another preferred embodiment, the microemulsion includes only one nonionic fatty alcohol surfactant.

In a preferred embodiment, the surfactant (such as a polyfluorocarbon surfactant or fatty alcohol surfactant) is soluble in both the perfluorocarbon and the aqueous medium.

In one embodiment, the microemulsion has a droplet size of less than about 100 nm, such as from about 5 to about 40 nm.

Another embodiment is a method of preparing a microemulsion of a perfluorocarbon comprising the steps of (a) mixing a polyfluorocarbon surfactant and an aqueous medium, (b) adding a perfluorocarbon (such as perflubron) to form the microemulsion.

### BRIEF DESCRIPTION OF THE DRAWINGS

FIG. 1 is a graph of a microemulsion titration comparing the volume of continuous phase to the total volume of co-surfactant.

FIG. 2 is a graph showing the percent transmittance (clarity) for (1) an oily liquid containing n-decane, 1% NaCl, and various nonylphenol surfactants varying in the degree of ethoxylation, and (2) the aqueous liquid containing 1% NaCl and various nonylphenol surfactants varying in the degree of ethoxylation.

### DETAILED DESCRIPTION OF THE INVENTION

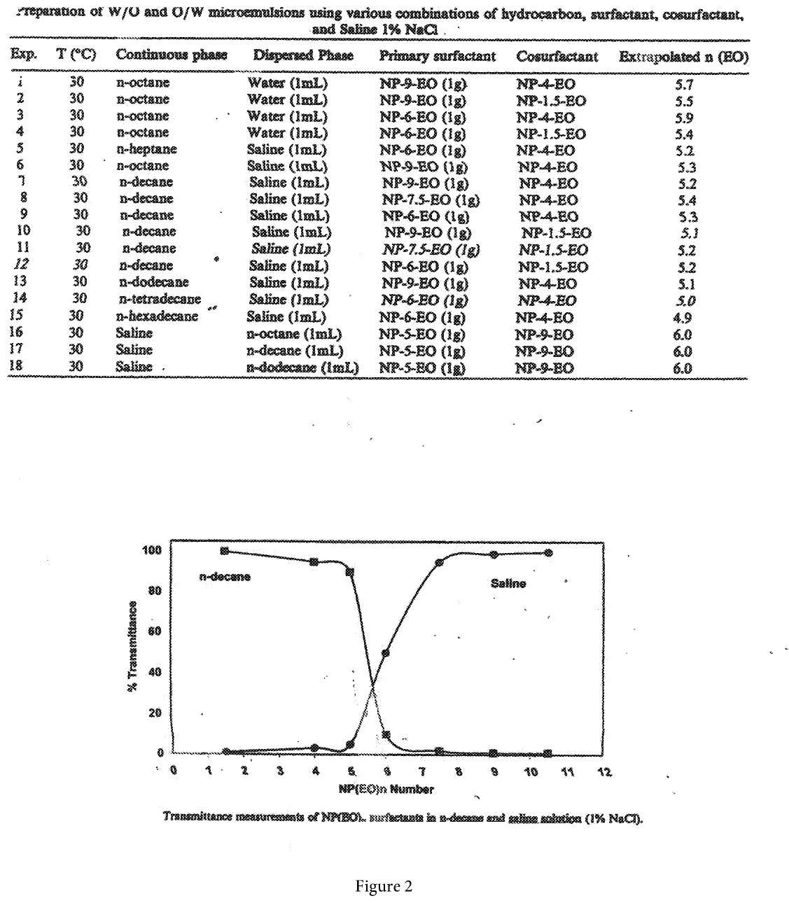

Water/Oil (W/O) and Oil/Water (O/W) microemulsions have been prepared with nonylphenol ethylene oxide NP(EO)ₙ surfactant. The preparation of an initial surfactant and a cosurfactant, both NP(EO)ₙ but with different “n” values, is based on two U.S. Pat. No. 3,778,381 issued on Dec. 11, 1972, and U.S. Pat. No. 4,146,499 issued on Mar. 27, 1979. Table 1 shown in FIG. 2 results from 18 different experiments that produced stable microemulsions.

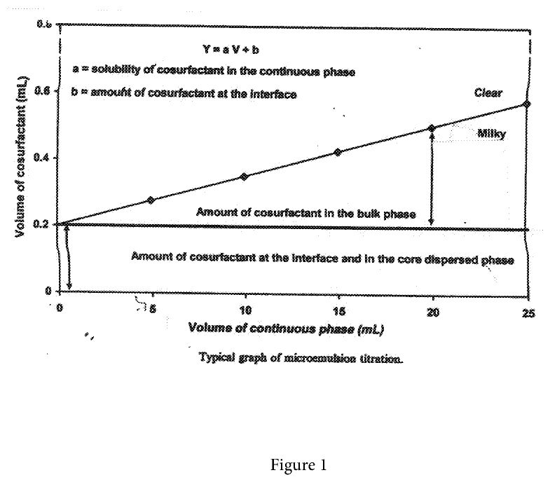

FIG. 1 is a typical graph of a microemulsion system titration. In this figure, the five data points represent five separate titrations whereby a cosurfactant was titrated into a fixed volume of the continuous phase (i.e., 5, 10, 15, 20 and 25 ml) until the solution transitioned from “milky” to “clear.” Extrapolation of the cosurfactant volume to zero volume of the continuous phase was used to determine the number of EO units (“n”) per molecule at the interface. This can be determined for each experiment as an average of the number of moles of ethoxylated units contained in the quantity of primary surfactant and cosurfactant at zero volume of the continuous phase. The extrapolated values for n determined in the 18 experiments were in the range of 5.5 to 6, and this value of n is that which achieves a balanced single surfactant microemulsion in which the surfactant is approximately equally soluble in both the oil and aqueous phases.

FIG. 2 shows the solubility of NP(EO)ₙ surfactant in two solutions, one a decane based O/W solution and one a saline based W/O solution. For n values ranging between 5 and 6, a domain of equal transmittance exists in both the oil phase and the aqueous phase. Where the NP(EO)ₙ surfactant in the interfacial film around the microdroplets is approximately equally soluble in the two phases, a stable microemulsion will form spontaneously.

Using similar methods with perfluorooctylbromide (PFOB) dispersed in a continuous phase of water or saline allows for the preparation of stable microemulsions using only a single ethoxylated surfactant.

A microemulsion is a low viscosity, isotropic, thermo-dynamically stable dispersion of two liquids (one being the “dispersed” phase, the other the “continuous” phase), in which the individual droplets of the dispersed phase generally have an average diameter that is smaller than ¼ of the wavelength of the visible light. These systems are distinctive in many ways, not only because of the transparency but also because of the small size of the dispersed phase droplets, which in practice typically range in diameter between 5 and 40 nm.

Microemulsions have several characteristics that make them the ideal vehicle to serve as a blood substitute, including:
1) a viscosity comparable to blood,
2) thermo-dynamic stability,
3) PFOB and other perfluorocarbons contribute to high degrees of oxygen solubilization,
4) single nonionic fatty alcohol surfactants are non-toxic, and
5) the average particle size of the droplets of the emulsion is typically less than 100 nm, which is small enough to pass through capillaries and, due to their small size, will circulate for a longer period of time in the body than larger particles.

For the reasons cited above, microemulsions of the present invention are useful as a blood substitute and have a reduced toxicity compared to prior emulsions systems.

### Determination of Effective Amount of Single Surfactant

A suitable amount and type of single surfactant for a microemulsion (oil-in-water or water-in-oil microemulsion) can be determined as follows.

A microemulsion is prepared containing an oily liquid (e.g., dodecane), a surfactant (e.g., nonylphenol 5 ethylene oxide, NP-5EO), and an aqueous liquid (e.g., saline). The mixture is stirred and then titrated with a second surfactant (a co-surfactant) (e.g., NP-9EO) until the mixture becomes clear. Additional aqueous liquid is added to the mixture and the mixture is titrated again with the second surfactant until the mixture becomes clear. This procedure is repeated, and the results of the microemulsion titration are plotted. An example of such a plot is provided in FIG. 1. Extrapolating the volume of co-surfactant to the value corresponding to a zero-volume continuous phase allows for the determination of the surfactant molar composition (e.g., the average number of ethylene oxide per nonylphenol absorbed at the interface between the two phases). The technique described in N. Naouli and H. Rosano, J. Dispersion of Science and Technology, 30:1-9, 2009, which is incorporated by reference, can be used.

The appropriate degree of ethoxylation for a surfactant (e.g., nonylphenol ethylene oxides) can be determined by (1) preparing oily liquids (e.g., n-decane with 1% NaCl), each containing the same amount of surfactant but with different degrees of ethoxylation, (2) similarly preparing aqueous liquids (e.g., saline with 1% NaCl), each containing the same amount of surfactant but with different degrees of ethoxylation, (3) measuring the transmittance of each, and (4) graphing the results as shown in FIG. 2. The intersection between the graphs for the oily liquids and aqueous liquids represents the appropriate degree of ethoxylation for a single surfactant microemulsion containing the oily liquid and aqueous liquid. When this degree of ethoxylation is incorporated into the single surfactant, the interfacial film molecules will be approximately equally soluble in both phases, and the system spontaneously forms a stable microemulsion.

U.S. Pat. No. 4,146,499, which is incorporated by reference, describes a method for determining the minimum amount of primary surfactant needed for a particular microemulsion system. In this theoretical calculation, the minimum amount of primary surfactant is found by calculating the surface area or, more readily, by calculating the monomolecular interfacial film area that must be formed between the dispersed phase and the continuous phase.

### Perfluorocarbon Microemulsion

One embodiment of the present invention is directed to a microemulsion of perfluorocarbon (e.g., PFOB) which includes a single surfactant system. The microemulsion includes perfluorocarbon dispersed within a continuous liquid phase. The microemulsions of the present invention are useful as blood substitutes and oxygen therapeutics.

One embodiment is a microemulsion comprising (a) perfluorocarbon, (b) aqueous medium; and (c) a non-ionic surfactant, such as a nonionic fatty alcohol surfactant or a nonionic polyfluorocarbon surfactant. In one preferred embodiment, the microemulsion includes no surfactant other than a polyfluorocarbon surfactant. In another preferred embodiment, the microemulsion includes only one polyfluorocarbon surfactant. In yet another embodiment, the microemulsion includes no surfactant other than a nonionic fatty alcohol surfactant. In yet another preferred embodiment, the microemulsion includes only one nonionic fatty alcohol surfactant.

Without being bound by any particular theory, the inventor believes that all the nonionic surfactant surrounds the nanoparticles and contributes to their stability and the solubilization of O₂ and CO₂.

### Perfluorocarbon

Suitable perfluorocarbons include, but are not limited to, perfluoro(tert-butylcyclohexane), perfluorodecalin, perfluoroisopropyl-decalin, perfluoro-tripropylamine, perfluorotributylamine, perfluoro-methylcyclohexylpiperidine, perfluoro-octylbromide, perfluoro-decylbromide, perfluoro-dichlorooctane, perfluorohexane, dodecafluoro-pentane, or a mixture thereof. In one preferred embodiment, the perfluorocarbon is perfluorooctylbromide (PFOB, aka, perflubron).

In one embodiment, the emulsion comprises from about 20 to about 80% w/v perfluorocarbon. In another embodiment, the emulsion comprises from about 40 to 70% w/v perfluorocarbon.

### Aqueous Medium

Suitable aqueous medium include, but are not limited to, saline, saline solutions (e.g., a 1% saline solution), water for injection, and 5% dextrose solution.

In one embodiment, the microemulsion comprises from about 40 to about 80% w/v aqueous medium (e.g., water for injection). In another embodiment, the microemulsion comprises from about 50 to about 70% w/v aqueous medium.

### Nonionic Surfactant

Suitable polyfluorocarbon surfactants include, but are not limited to, poly(ethoxylated) fluorocarbon surfactants, poly(propoxylated) fluorocarbon surfactants, and mixtures thereof. In a preferred embodiment, the polyfluorocarbon surfactant is soluble in both the perfluorocarbon and the aqueous medium. In one embodiment, the polyfluorocarbon surfactant has a perfluorocarbon moiety.

The polyfluorocarbon surfactant can have the formula (F₃C)—(CF₂)ₐ—(CH₂)_{b}—(OCH₂CH₂)_{c}—OH, where the variables ‘a’, ‘b’, and ‘c’ represent the relative quantities of each chemical group (CF₂, CH₂, and OCH₂CH₂). For instance, the variable ‘a’ can range from 1 to 20, the variable ‘b’ ranges from 1 to 20, and the variable ‘c’ ranges from 1 to 20. In one embodiment, ‘a’ is from 3 to 5, ‘b’ is 2, and ‘c’ is from 3 to 5. In another embodiment, ‘a’ is 3, ‘b’ is 2, and ‘c’ is about 4.4. In yet another embodiment, ‘a’ is 5, ‘b’ is 2, and ‘c’ is about 4.4. In yet another embodiment, ‘a’ is 5, ‘b’ is 2, and ‘c’ is about 4.7. In yet another embodiment, the variable ‘c’ ranges from 2 to 3.

Suitable fatty alcohol surfactants include those having the formula CH₃—(CH₂)_{b}—(OCH₂CH₂CH₂)_{c}—OH, where the variables ‘b’ and ‘c’ represent the relative quantities of each chemical group (CH₂ and OCH₂CH₂CH₂). For instance, the variable ‘b’ ranges from 1 to 20 and the variable ‘c’ ranges from 1 to 20. In one embodiment, the variable ‘b’ ranges from 9 to 11 and the variable ‘c’ ranges from 5 to 7, such as from 6 to 7. For example, the single fatty alcohol surfactant can be C₁₁(EO)_{6.4}.

In one embodiment, the microemulsion comprises from about 1 to about 10% w/v nonionic surfactant. In another embodiment, the microemulsion comprises from about 2 to about 6% w/v nonionic surfactant.

### Microemulsion

The microemulsion may also include pharmaceutically acceptable excipients, such as isotonicity agents, buffers, vitamins (e.g., vitamin E), antibiotics, and any combination of any of the foregoing.

In one embodiment, the microemulsion is isotonic.

In another embodiment, the aqueous medium is buffered to a pH of 6.8-7.4.

In one embodiment, the microemulsion has a droplet size of less than about 100 nm, such as from about 5 to about 50 nm or from about 5 to about 40 nm. In an embodiment, 90% or more of the total amount by volume of the dispersed particles have a droplet size of less than 40 nm.

The particle size distribution can be determined using a laser light scattering particle-size distribution analyzer.

The microemulsion may be prepared by (a) mixing a non-ionic polyfluorocarbon surfactant and an aqueous medium, and (b) adding perfluorocarbon (e.g., PFOB) to form the microemulsion. The mixture can be mixed at, for instance, 2,000 to 7,000 rpm or higher. The mixture can be homogenized. In one embodiment, the homogenization is performed under high pressure.

The microemulsion can be administered, for instance, topically, parenterally, intravenously or intra-arterially. The microemulsion can be diluted in a physiological solution prior to, for instance, parenteral, intravenous, or intraarterial administration.

### Uses

Another embodiment is a method of treating sickle cell disease, decompression sickness, air embolism or carbon monoxide poisoning in a subject suffering therefrom comprising administering to the subject the microemulsion described herein effective to treat the subject's sickle cell disease, decompression sickness, air embolism or carbon monoxide poisoning. In one embodiment, the microemulsion is administered intravenously (IV) or intrathecally.

“Carbon monoxide poisoning” includes the poisoning of a subject resulting from exposure to carbon monoxide. Toxicity of carbon monoxide can vary with the length of exposure, concentration of CO that the subject was exposed to, respiratory and circulatory rates. Symptoms of carbon monoxide poisoning can vary with the percent carboxyhemoglobin present in the blood and can include headache, vertigo, dyspnea, confusion, dilated pupils, convulsions and coma (some of which result from injury to the brain). A standard treatment for carbon monoxide poisoning is the administration of 100% oxygen by breathing mask (The Merck Manual, 1999; Prockop, 2007).

Yet another embodiment is a method of preserving an organ prior to transplant comprising contacting the organ with the microemulsion described herein effective to increase the organ's survival time. In one embodiment, the organ is perfused with the microemulsion.

Yet another embodiment is a method of treating a wound, a burn injury, acne or rosacea in a subject suffering therefrom comprising topically administering to the skin of the subject the microemulsion described herein effective to treat the subject's wound, burn injury, acne or rosacea. The term “burn injury” includes a wound resulting from a burn injury, which is a first, second or third degree injury caused by thermal heat, radiation, electric or chemical heat, for example as described at page 2434, section 20, chapter 276, of The Merck Manual, 17.sup.th Edition (1999), Merck Research Laboratories, Whitehouse Station, N.J., U.S.A.

Yet another embodiment is a method of increasing the firmness of the skin or reducing the appearance of fine lines, wrinkles or scars in a subject comprising topically administering to the skin of the subject the microemulsion described herein effective to increase the firmness of the subject's skin or reduce the appearance of fine lines, wrinkles or scars on the subject's skin.

### EXAMPLES

### Example 1

### Preparation of Microemulsion

A polyfluorocarbon surfactant of the formula (F₃C)—(CF₂)₃—(CH₂)₂—(OCH₂CH₂)_{4.4}—OH and an aqueous medium are mixed at an appropriate ratio. The aqueous medium is a 1% saline solution of a 5% dextrose solution. Perflubron is added and mixed.

### Example 2

The procedure in Example 1 was repeated using the polyfluorocarbon surfactant (F₃C)—(CF₂)₅—(CH₂)₂—(OCH₂CH₂)_{4.4}—OH.

### Example 3

The procedure in Example 1 was repeated using the polyfluorocarbon surfactant (F₃C)—(CF₂)₅—(CH₂)₂—(OCH₂CH₂)_{4.7}—OH.

### Example 4

When the procedure in Example 1 is repeated using 1 ml of perfluoro-octyl-bromide (PFOB) (dispersed phase) in 1% saline (continuous phase) and fatty alcohol C₁₁(EO)₅ (primary surfactant) and C₁₁(EO)₇ (co-surfactant) at 30° C., it produces a microemulsion. (“EO” refers to ethylene oxide.) Plotting the volume of co-surfactant versus the volume of the aqueous continuous phase, and extrapolating to zero volume of the continuous phase, shows that a single surfactant microemulsion with C₁₁(EO)_{6.4} can be produced. See FIG. 1.

The stability of the microemulsions prepared in Examples 1-3 are determined. The results are provided in the table below.

| Surfactant | Range of Stability (° C.) |
|---|---|
| (F₃C)-(CF₂)₃-(CH₂)₂-(OCH₂CH₂)_{4.4}-OH | 19-45 |
| (F₃C)-(CF₂)₅-(CH₂)₂-(OCH₂CH₂)_{4.4}-OH | 13-53 |
| (F₃C)-(CF₂)₅-(CH₂)₂-(OCH₂CH₂)_{4.7}-OH | 22-54 |

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above. It is intended that the appended claims define the scope of the invention and that methods and structures within the scope of these claims and their equivalents be covered thereby.

All publications and patent and/or patent applications cited in this application are herein incorporated by reference to the same extent as if each individual publication or patent application was specifically and individually indicated to be incorporated herein by reference.

1. A single-surfactant microemulsion comprising:
(a) an oil;
(b) aqueous medium; and
(c) a polyfluorocarbon surfactant, which is soluble in both the oil and aqueous medium.

2. The microemulsion of claim 1, wherein the oil is perflubron.

3. The microemulsion of claim 1, wherein the microemulsion includes no surfactant other than the polyfluorocarbon surfactant.

4. The microemulsion of claim 1, wherein microemulsion includes only one polyfluorocarbon surfactant.

5. The microemulsion of claim 1, wherein the aqueous medium is saline.

6. The microemulsion of claim 1, wherein the aqueous medium is a 5% dextrose solution.

7. The microemulsion of claim 1, wherein the polyfluorocarbon surfactant is soluble in both the polyfluorocarbon and the aqueous medium.

8. The microemulsion of claim 1, wherein the polyfluorocarbon surfactant is a poly(ethoxylated) fluorocarbon surfactant.

9. The microemulsion of claim 1, wherein the poylfluorocarbon surfactant has the formula (F₃C)—(CF₂)ₐ—(CH₂)_{b}—(OCH₂CH₂)_{c}—OH, wherein the variables ‘a’, ‘b’, and ‘c’ represent the relative quantities of each chemical group.

10. The microemulsion of claim 9, wherein ‘a’ is from 3 to 5, ‘b’ is 2, and ‘c’ is from 3 to 5.

11. The microemulsion of claim 9, wherein ‘a’ is 3, ‘b’ is 2, and ‘c’ is about 4.4.

12. The microemulsion of claim 9, wherein ‘a’ is 5, ‘b’ is 2, and ‘c’ is about 4.4.

13. The microemulsion of claim 9, wherein ‘a’ is 5, ‘b’ is 2, and ‘c’ is about 4.7.

14. The microemulsion of claim 1, wherein the polyfluorocarbon surfactant is a poly(propoxylated) fluorocarbon surfactant.

15. The microemulsion of claim 1, wherein the polyfluorocarbon surfactant has the formula (F₃C)—(CF₂)ₐ—(CH₂)_{b}—(OCH₂CH₂CH₂)_{c}—OH, wherein the variables ‘a’, ‘b’, and ‘c’ represent the relative quantities of each chemical group.

16. The microemulsion of claim 1, wherein the oil comprises a perfluorocarbon selected from perfluoro(tert-butylcyclohexane), perfluorodecalin, perfluoroisopropyldecalin, perfluoro-tripropylamine, perfluorotributylamine, perfluoro-methylcyclohexylpiperidine, perfluorooctylbromide, perfluoro-decylbromide, perfluoro-dichlorooctane, perfluorohexane, dodecafluoropentane, or any combination of any of the foregoing.

17. The microemulsion of claim 16, wherein the perfluorocarbon is perfluorooctylbromide.

18. The microemulsion of claim 1, wherein the microemulsion has a droplet size of less than about 100 nm.

19. The microemulsion of claim 1, wherein the microemulsion has a droplet size of from about 5 to about 40 nm.

20. A blood substitute comprising the microemulsion of claim 1.

21. A method of preparing a microemulsion of perflubron comprising the steps of:
(a) mixing a polyfluorocarbon surfactant and an aqueous medium,
(b) adding perflubron to form the microemulsion,
wherein the polyfluorocarbon surfactant is soluble in the aqueous medium and perflubron.
